(19) United States
(12) Patent Application Publication
(10) Pub. No.: US 2023/0398190 A1
(43) Pub. Date: Dec. 14, 2023

(54) COMPOSITIONS AND METHODS FOR TREATING SOLID CANCER

(71) Applicant: EXOXEMIS, INC., Little Rock, AR (US)

(72) Inventors: Robert C. Allen, Omaha, NE (US); Jackson T. Stephens, Jr., Little Rock, AR (US)

(21) Appl. No.: 18/035,480

(22) PCT Filed: Oct. 1, 2021
(86) PCT No.: PCT/US2021/053155

§ 371 Date: May 4, 2023

Related U.S. Application Data
(60) Provisional application No. 63/112,447, filed on Nov. 11, 2020.

(51) Int. Cl. A61K 38/44; A61K 33/40; A61K 33/20; A61K 47/26; A61P 35/00

(52) U.S. Cl. CPC A61K 38/443; A61K 33/40; A61K 33/20; A61K 47/26; A61P 35/00; C12Y 111/01007

(57) ABSTRACT

Compositions and methods for treating solid cancer are provided. Specifically, the disclosure provides compositions comprising haloperoxidases, and methods comprising administering such compositions, for treating solid cancer.

### BACKGROUND

The present disclosure generally relates to compositions and methods for treating solid cancer. Specifically, the disclosure provides compositions comprising haloperoxidases, and methods comprising administering such compositions, for treating solid cancer.

There is no doubt that cancer is and will remain a major impact on society, globally. According to the current statistics produced by the United States National Institutes of Health, in 2020, an estimated 1,806,590 new cases of cancer will be diagnosed in the United States and 606,520 people will die from the disease (https://www.cancer.gov/about-cancer/understanding/statistics).

Cancer is a disease that is characterized by uncontrolled cell growth, almost anywhere in the body. Tumor formation is where uncontrolled cell growth occurs in solid tissue such as an organ, muscle, or bone. To this point, a large portion of the most common cancers are solid, tumor-forming cancers such as breast cancer, lung and bronchus cancer, prostate cancer, colon and rectum cancer, melanoma of the skin, bladder cancer, kidney and renal pelvis cancer, endometrial cancer, pancreatic cancer, thyroid cancer, and liver cancer. Solid cancers include various cancers other than hematological cancers (lymphoma, leukemia, and multiple myeloma etc).

While rates of cancer survival are increasing through the development of improved therapies, the cancer mortality rate remains high—158.3 deaths per 100,000 men and women per year in the United States (based on 2013-2017 deaths). Improved cancer therapies are therefore the subject of ongoing research and development by the scientific community.

For most solid cancers, abnormal tissue is biopsied for diagnosis. Surgery to reduce the size of, or eradicate a cancer, (commonly referred to as debulking) may be a therapeutic option. Debulking may also increase the effectiveness of subsequently administered anticancer therapies, such as immunotherapy, chemotherapy and/or radiotherapy. However, surgical intervention in cancer therapy (whether by biopsy or debulking) is not without risk. In addition to reproducing uncontrollably, cancer cells lose cohesiveness and organization of normal tissue, and may detach from a primary tumor during biopsy or surgery to travel elsewhere in the body via the circulatory and lymphatic systems. Cancer spread (i.e. metastases) during biopsy or surgical intervention therefore presents a significant risk to a cancer patient.

Some solid cancers, such as bladder, brain or spinal cord cancer, are difficult to biopsy and/or treat (surgically or non-surgically) through inaccessibility to the site of cancer growth. Accordingly, such cancers may result in high incidences of patient mortality.

There is a need to minimize solid cancer metastasis during biopsy or surgery, and/or to improve treatment of ‘hard-to-reach’ solid cancers.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure is predicated on the surprising and unexpected finding that haloperoxidase-containing compositions exhibit anticancer properties.

According to one aspect of the disclosure, there is provided a method of treating a solid cancer comprising administering an effective amount of a pharmaceutical composition comprising a haloperoxidase.

In another aspect, the disclosure provides a method of treating a solid cancer in a patient, said method consisting of administering to said patient an effective amount of a haloperoxidase, and optionally one or more of: a halide, a peroxide or peroxide producing oxidase, a substrate for said oxidase, and a pharmaceutically acceptable carrier.

In yet another aspect, the disclosure provides a combination for treating a solid cancer in a patient, said combination comprising a haloperoxidase, and at least one of a halide, and peroxide or a peroxide producing oxidase.

In yet another aspect, the disclosure provides a combination for treating a solid tumor in a patient, said combination consisting of a haloperoxidase, a halide, and peroxide or a peroxide producing oxidase, and optionally a substrate for said oxidase, and a pharmaceutically acceptable carrier.

In yet another aspect, the disclosure provides a composition for treating a solid cancer in a patient, said composition comprising a haloperoxidase, and optionally one or more of: a halide, peroxide or a peroxide producing oxidase, a substrate for said oxidase, and a pharmaceutically acceptable carrier.

In yet another aspect, the disclosure provides a composition for treating a solid cancer in a patient, said composition consisting of a haloperoxidase, and optionally one or more of a halide, peroxide or a peroxide producing oxidase, a substrate for said oxidase, and a pharmaceutically acceptable carrier.

In embodiments, the haloperoxidase is selected from a group consisting of: myeloperoxidase (MPO), eosinophil peroxidase (EPO), lactoperoxidase (LPO), chloroperoxidase (CPO), functional derivatives thereof, and combinations thereof. Most preferably, the haloperoxidase is EPO.

In embodiments, the haloperoxidase catalyzes halide oxidation and disproportionation of peroxide yielding singlet molecular oxygen resulting in one or more of: inhibition of cancer cell growth, inhibition of cancel cell metastases, and/or cancer cell death.

In embodiments, the solid cancer is selected from the group consisting of: breast cancer, lung and bronchus cancer, prostate cancer, colon and rectum cancer, melanoma of the skin, bladder cancer, kidney and renal pelvis cancer, endometrial cancer, pancreatic cancer, thyroid cancer, liver cancer, brain cancer and spinal cord cancer.

Other embodiments of the invention will be evident from the following detailed description of various aspects of the invention

### DETAILED DESCRIPTION

### Definitions

The terms “a,” “an,” “the” and similar referents used in the context of describing inventive concepts (especially in the context of the following claims) are to be construed to cover both the singular and the plural, unless otherwise indicated herein or clearly contradicted by context.

All methods described herein can be performed in any suitable order unless otherwise indicated herein or otherwise clearly contradicted by context.

The use of any and all examples, or exemplary language (e.g., “such as”) provided herein is intended merely to provide better illumination and does not pose a limitation on the scope of the disclosure. No language in the specification should be construed as indicating any non-claimed element is essential.

Except where otherwise indicated, all numbers expressing quantities of ingredients, reaction conditions, and so forth used in the specification and claims are to be understood as being modified in all instances by the term ‘about’. Accordingly, unless indicated to the contrary, the numerical parameters set forth in the following specification and attached claims are approximations that may vary depending upon the desired properties sought to be obtained by the present disclosure. At the very least, each numerical parameter should be construed in light of the number of significant digits and ordinary rounding conventions. The term “about” may be understood to refer to a range of +/−10%, such as +/−5% or +/−1% or, +/−0.1%.

Recitation of ranges of values herein is merely intended to serve as a shorthand method of referring individually to each separate value falling within the range. Unless otherwise indicated herein, each individual value is incorporated into the specification as if it were individually recited herein. For example, if a range is from about 1 to about 50, it is deemed to include, for example, 1, 7, 34, 46.1, 23.7, or any other value or range within the range.

The terms “protein” and “polypeptide” are used interchangeability herein. The 3-letter code for amino acids as defined in conformity with the IUPAC-IUB Joint Commission on Biochemical Nomenclature is used throughout this disclosure. It is also understood that a polypeptide may be coded for by more than one nucleotide sequence due to the degeneracy of the genetic code.

Reference is made herein to “enzymes”, such as haloperoxidase or glucose oxidase. In the present context, an enzyme is a protein/polypeptide which acts as a catalyst to bring about a specific biochemical reaction. Included within the scope of enzymes of the present disclosure include those isolated from a natural source having the unmodified amino acid sequence identical to that found in nature, as well as “functional derivatives” thereof.

The term “haloperoxidase” refers to an enzyme which catalyzes the hydrogen peroxide dependent oxidation of halide generating hypohalous acid; this hypohalous acid can react with an additional hydrogen peroxide to generate singlet molecular oxygen. A haloperoxidase according to the present disclosure may be also referred to as a halide:hydrogen peroxide oxidoreductase (e.g., EC No, 1.11.1.7 and EC No. 1.11.1.10 under the International Union of Biochemistry) for which halide, e.g., chloride or bromide, is the electron donor or reductant and peroxide is the electron receiver or oxidant. Suitable haloperoxidases, include myeloperoxidase (MPO), eosinophil peroxidase (EPO), lactoperoxidase (LPO), chloroperoxidase (CPO), functional derivatives thereof and combinations thereof. The haloperoxidase may be derived from any source, including human and non-human animals.

A “derivative” of an enzyme of the disclosure generally retains the characteristic enzymatic activity observed in the wild-type, native or parent form to the extent that the derivative is effective for similar purposes as the wild-type, native or parent form.

The term “functional fragment” or “functional derivative” when used in the contact of enzymes of the disclosure encompasses naturally occurring, synthetically or recombinantly produced nucleic acids or fragments and encode enzymes having the functional characteristics of the native, unmodified parent enzyme present disclosure. A “functional derivative” may include a “substituted variant” which is a variant in which at least one amino acid residue in a native sequence has been removed and inserted into the same position by a different amino acid. The substitution may be single, wherein only one amino acid in the molecule is substituted; or there may be multiple, wherein the same molecule has two or more amino acids substituted. Multiple substitutions can be located at successive sites. Likewise, an amino acid can be substituted with multiple residues, including substitutions and insertions. An “insertion variant” is a variant in which one or more amino acids are inserted into an amino acid immediately adjacent to a particular position in a native sequence. Immediately adjacent to the amino acid means attached via an alpha-carboxy or alpha-amino functional group of the amino acid, A “deleted variant” is a variant in which one or more amino acids in the native amino acid sequence are removed. Typically, a deleted variant has one or two amino acids deleted in a particular region of its molecule.

The term “isolated” or “purified” refers to a material that is removed from its original environment (e.g, the natural environment, if it is naturally occurring). For example, the material is said to be “purified” when it is present in a particular composition in a higher concentration than exists in a naturally occurring or wild type organism or in combination with components not normally present upon expression from a naturally occurring or wild type organism. For example, a naturally-occurring protein/polypeptide present in a living organism is not isolated, but the same protein/polypeptide, separated from some or all of the coexisting materials in the natural system, is isolated. Such proteins/polypeptides could, for example, be part of a composition, and still be isolated in that such a composition is not part of the natural environment of the proteins/polypeptides.

The term “pharmaceutically acceptable” as used herein refers to substances that do not cause substantial adverse allergic or immunological reactions when administered to a patient. A “pharmaceutically acceptable carrier” includes, but is not limited to, solvents, coatings, dispersion agents, wetting agents, isotonic and absorption delaying agents and disintegrants.

As used herein, “treat”, “treating” or “treatment” of a disease, condition or disorder means accomplishing one or more of the following: (a) reducing the severity and/or duration; (b) limiting or preventing development of characteristic symptoms; (c) inhibiting worsening of symptoms; (d) limiting or preventing recurrence; and (e) limiting or preventing recurrence of symptoms. That is, the terms include both prophylactic or preventive treatment (that prevent and/or slow the development of a targeted pathologic disease, condition or disorder) and curative, therapeutic or disease-modifying treatment, including therapeutic measures that cure, slow down, lessen symptoms of, and/or halt progression of a disease, condition or disorder; and treatment of a patient at risk of contracting a disease or suspected to have contracted a disease, as well as a patient who is ill or has been diagnosed as suffering from a disease, condition or disorder. The terms do not necessarily imply that a patient is treated until total recovery. The terms may also refer to the maintenance and/or promotion of health in an individual not suffering from a disease but who may be susceptible to the development of an unhealthy condition. The terms may also include the potentiation or otherwise enhancement of one or more primary prophylactic or therapeutic measures. As non-limiting examples, a treatment can be performed by a patient, a caregiver, a doctor, a nurse, or another healthcare professional.

As used herein, “prevent”, “preventing”, “prevention”, or “prophylaxis” of a disease or disorder means preventing that a disorder occurs in patient. “Prevention” includes reduction of risk, incidence and/or severity of a disease, condition or disorder.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

The terms “effective amount” or “therapeutic amount” are intended to mean that amount of a substance that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

The terms “comprise”, “comprises”, “comprised” or “comprising”, “including” or “having” and the like in the present specification and claims are used in an inclusive sense, that is to specify the presence of the stated features but not preclude the presence of additional or further features.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

### Haloperoxidase-Containing Compositions.

Haloperoxidases are widespread in nature being produced by mammals, plants, algae, lichen, bacteria, and fungi. PCT/US19921001237 discloses that haloperoxidases can be used as an antimicrobial agents (effective particularly against bacteria and fungi) as they selectively bind to target microbes and in the presence of peroxide and halide inhibiting target microbe growth. Using limited concentrations of haloperoxidase with selective binding can inhibit target microbes without eliminating desirable microbes or causing significant damage to host cells. The selective nature of haloperoxidase binding makes them useful in therapeutic or prophylactic antimicrobial treatment of human or non-human patients.

The present disclosure is predicated on the surprising and unexpected finding that haloperoxidase-containing compositions exhibit anticancer properties. In one aspect, the disclosure provides methods for treating solid cancer by contacting the cancer with a composition comprising a haloperoxidase. In another aspect, the disclosure provides compositions for treating solid cancer, said compositions comprising a haloperoxidase. In yet another aspect, the disclosure provides a combination for treating a solid cancer, said combination comprising a haloperoxidase, and at least one of a halide, and peroxide or a peroxide producing oxidase.

In some embodiments, the haloperoxidase catalyzes halide oxidation and disproportionation of peroxide to singlet molecular oxygen treating said cancer by inhibiting cancer cell growth, metastases and/or by cancer cell killing. In embodiments, suitable haloperoxidases according to the present disclosure include eosinophil peroxidase (EPO), myeloperoxidase (MPO), lactoperoxidase (LPO), chloroperoxidase (CPO), functional derivatives thereof and combinations thereof.

In other embodiments, the method of treatment of the present disclosure further comprises administering an effective amount of peroxide or a peroxide-producing oxidase. A substrate for the oxidase may be optionally administered. Preferably, the peroxide-producing oxidase is glucose oxidase and the substrate is glucose. In further embodiments, the method further comprises administering the haloperoxidase with a halide, preferably a chloride or bromide.

In yet other embodiments, the haloperoxidase is administered in a first composition together with at least one further composition comprising one or more of: a halide, peroxide or a peroxide-producing oxidase plus a substrate for the peroxide-producing oxidase. Alternatively, the haloperoxidase may be formulated in a composition for administration, said composition also comprising one or more of a halide, peroxide or a peroxide-producing oxidase, and a substrate for the peroxide-producing oxidase.

In particular embodiments, eosinophil peroxidase (EPO) and myeloperoxidase (MPO) are preferred haloperoxidases for use in the present compositions, combinations and treatment methods. In further embodiments, MPO and EPO are porcine derived. Preferably, the purified haloperoxidases, porcine MPO and EPO, are those produced by Exoxemis, Inc. The porcine MPO is preferably 98.9% pure by ultraperformance liquid chromatography (RP-UPLC) and 100% pure by molecular size exclusion high-performance liquid chromatography (SEC-HPLC). The guaiacol unit (GU) activity of the porcine MPO is preferably 404 GU/mg; 1.0 GU of activity consumes 1.0 μmol H₂O/minute.

Porcine EPO is preferably 99.2% pure by reversed-phase high-performance liquid chromatography. The guaiacol unit (GU) activity of the porcine EPO is preferably 80 GU/mg.

MPO and EPO are both cationic proteins, Without being bound by theory, it is believed that the cationic nature of such haloperoxidases makes them particularly adherent to the anionic surface of cancer cells resulting from the Warburg effect (a form of modified cellular metabolism found in cancer cells). Thus, the anticancer effect results from the electrostatic attraction and binding of the haloperoxidase to the anionic surface of cancer cells, but not to the neutrally-charged surface of normal cells.

Haloperoxidases may differ in their physical properties and optimal conditions for enzymic activity (e.g. see U.S. Pat. No. 9,782,459). For example, MPO is around 150 kDa and is active at acidic pH (4.0-6.5), whereas EPO is around 70 kDa and active at acidic to neutral pH (i.e. 6.5-7.4). Notwithstanding the above surprising and unexpected finding that haloperoxidases have anticancer potential, in embodiments, compositions of the present disclosure may comprise one or more haloperoxidase where the characteristics of the haloperoxidase may be aligned with the conditions of the site of cancer treatment. In embodiments, the choice of haloperoxidase is determined by the pH at the site of treatment. In other embodiments, the choice of haloperoxidase is determined by accessibility to the site of treatment.

Effective amounts of haloperoxidase employed in the compositions, combinations or treatment methods of the disclosure may vary widely depending on conditions under which the compositions are employed, the environment of use and the desired result. In some embodiments, the compositions of the disclosure will comprise from about 1 to about 100,000 μg/ml of haloperoxidase, more preferably from about 5 to about 50,000 μg/ml, and even more preferably from about 10 to about 5,000 μg/ml haloperoxidase.

Peroxide-producing oxidases effective in the present disclosure include, for example, oxidases, such as glucose oxidase, cholesterol oxidase and galactose oxidase. As a representative example, when the oxidase is glucose oxidase and its substrate is glucose, the compositions of the present disclosure may comprise from about 0.05 to about 3,000 U/ml, more preferably from about 0.1 to about 1,000 U/ml, and even more preferably from about 1 to about 500 U/ml of glucose oxidase, and from about 0.1 to about 100 mM, more preferably from about 0.5 to about 80 mM, and even more preferably from about 1 to about 50 mM glucose. Preferably, the glucose oxidase as used in compositions of the present disclosure is derived from Aspergillus niger. More preferably, the glucose oxidase is that produced by Exoxemis, Inc, which is isolated from Aspergillus niger, purified to 99.8% by RP-HPLC and 99.9% by SECHPLC, and optionally wherein the unit (U) activity of GO was 309 U/mg (in which 1.0 U oxidizes 1.0 μmol of β-D-glucose to D-gluconolactone and H₂O₂/minute at pH 5.1 at 35° C.

As noted above, haloperoxidases useful in the compositions, combinations or treatment methods of the present disclosure, if not used in combination with a peroxide-producing oxidase, may be administered in combination with peroxide. Administration of peroxide, as with a peroxide-producing oxidase, may be simultaneously or sequentially to the administration of the haloperoxidase. In embodiments, peroxide may be administered to a site of treatment at a concentration including, but not limited to, about 1 μM to about 100 mM, preferably about 1 mM to about 50 mM, more preferably about 9 mM. Administration may depend on accessibility to the site of treatment. In embodiments, a bolus of peroxide of between about 1 ml to 1000 ml, preferably 100 ml to 800 ml, most preferably 500 ml may be administered.

In some embodiments, the haloperoxidase may optionally be supplied to a site of treatment with at least two amino acids, preferably at least three amino acids, selected from the group consisting of glycine, L-alanine, D-alanine, L-alanine anhydride, L-glutamine, L-glutamic acid, glycine anhydride, hippuric acid, L-histidine, L-leucine, D-leucine, L-isoleucine, D-isoleucine, L-lysine, L-ornithine, D-phenylalanine, L-phenylalanine, L-proline, L-hydroxyproline, L-serine, taurine, L-threonine, D-threonine, L-tyrosine, L-valine, D-valine, beta amino acids, such as beta alanine, L-beta-homoleucine, D-beta-homoleucine, 3-aminobutanoic acid, L-2,3-diaminopropionic acid monohydrochloride, D-2,3-diaminopropionic acid monohydrochloride, L-3-aminoisobutyric acid, D-3-aminoisobutyric acid, ethyl 3-aminobutyrate, sarcosine methyl ester hydrochloride and nipecotic acid, or an alkyl ester or pharmaceutically acceptable salt thereof. In a particular embodiment, the haloperoxidase may be formulated together with the amino acids, or alternatively the amino acids may be supplied as a separate composition for premixing before administration, or simultaneous or concurrent administration.

Effective amounts of the amino acids which may be optionally employed in the compositions/combinations of the disclosure will vary depending on the amount of haloperoxidase in the compositions/combinations and conditions present in the environment of use. In an example, the compositions may generally comprise from about 0.1 to about 500 mM, more preferably from about 0.2 to about 100 mM, and even more preferably from about 0.3 to about 50 mM of each of the amino acids of the disclosure.

The compositions/combinations of the present disclosure may optionally comprise a halide. When the halide is chloride, the amount of chloride used in the compositions of the present disclosure will preferably fall in the range of about 10 μmol chloride to about 200 μmol per ml of solution (i.e., 10 to 200 mEq chloride/L) chloride. The physiologic concentration of chloride in plasma is about 105 mEq/L. When included, the compositions of the present disclosure may comprise from about 0.5 μmol bromide to about 20 paid bromide per ml (i.e., 0.5 to 20 mEq bromide/L) of liquid composition, more preferably from about 1 μmol bromide to about 10 μmol bromide per ml (i.e., 1 to 10 mEq bromide/L) of liquid composition, and most preferably from about 100 nmol bromide to about 1 μmol bromide per ml of liquid composition.

The compositions/combinations may optionally comprise a pharmaceutically acceptable carrier. In some embodiments, the compositions may be conveniently provided in a liquid carrier. Any liquid carrier may be generally used for this purpose, provided that the carrier does not significantly interfere with the selective binding capabilities of the myeloperoxidase or with enzyme activity. Alternatively, the compositions may be provided in solid form with activation on solubilization in liquid.

In embodiments of compositions or combinations of the present disclosure that include a substrate for the peroxide-producing oxidase, the haloperoxidase lends itself to construction as a binary formulation in which the composition's active agents are formulated in two separate parts for consolidation at the time of use. For example, the first composition of the binary formulation may comprise a solution containing both the haloperoxidase and the oxidase. In some embodiments, the first composition may optionally comprise two or three amino acids. In some embodiments, the three amino acids are glycine, 1-alanine and 1-proline. The second composition of the binary formulation may comprise a substrate for the oxidase, e.g., glucose (i.e., dextrose) in the case of glucose oxidase. The substrate may be provided, for example, in the form of a solid wafer. In some embodiments, the haloperoxidase composition may additionally comprise alcohol in order to facilitate oxidase substrate solubilization and utilization by the oxidase.

In one embodiment, the methods of the present disclosure comprise administering to a site, prophylactically or therapeutically, a combination of compositions. For example, a first composition comprising haloperoxidase and a peroxide-producing oxidase may be administered (optionally comprising at least two amino acids). A second composition comprising a substrate for the oxidase may be separate. In some embodiments, the first composition and the second composition are mixed before administration to the site of infection. In some embodiments the first composition and the second composition are administered concurrently to the site. In some embodiments the first composition and the second composition are administered sequentially to the site. The first composition and the second composition may be administered in any order.

As an illustrative example, a composition of the present disclosure suitable for use as anticancer treatment may comprise from about 1 to 50,000 μg/ml haloperoxidase, from 0.01 to 500 units of glucose oxidase, and optionally: from 0.1 to 500 μmol/mL (i.e., from 0.1 to 500 mM) of glycine, from 0.1 to 500 μmol; mL (i.e., from 0.1 to 500 mM) of D-isoleucine, from 0 to 100 μmol/mL (i.e., from 0 to 100 mM) of L-alanine, and from 50 to 500 mEq/L of chloride. The above composition may be combined with from 1 to 500 μmol/mL (i.e., from 1 to 500 mM) of glucose or dextrose.

### Treatable Cancers

The cancers targeted by the present invention are solid cancers and include various cancers other than hematological cancers (malignant lymphoma, leukemia, multiple myeloma etc). Typical examples of a solid cancer include lung cancer, breast cancer, stomach cancer, liver cancer, colon cancer, tongue cancer, thyroid cancer, kidney cancer, prostate cancer, uterine cancer, cervical cancer, and ovary. Preferred specific examples of the solid cancer include, for example, bladder cancer, colon cancer, lung cancer, pancreatic cancer, kidney cancer, or breast cancer. A solid cancer may also Include melanoma or glioma, but is not limited thereto.

In the context of non-surgical anticancer treatment, such as topical applications, anticancer compositions of the present disclosure can be administered in any effective pharmaceutically acceptable form to warm blooded animals, including human and non-human animal patients. In this context, compositions of the disclosure may be administered at any mucosal or epithelial surface. For example, the compositions of the disclosure may be administered in topical, lavage, oral, vaginal or rectal suppository dosage forms, as a topical, buccal, nasal spray, aerosol for inhalation or in any other manner effective.

For topical applications, the pharmaceutically acceptable carrier may take the form of liquids, creams, foams, lotions, ointments, suspensions, suppositories or gels, and may additionally comprise aqueous or organic solvents, buffering agents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration. In addition, the compositions of the present disclosure may be impregnated in dressings or coverings for application to a patient.

In the context of more invasive anticancer treatment, such as tumor debulking (size reduction or elimination by excision) by surgery, anticancer compositions of the present disclosure may be administered extratumorally or intratumorally, or a combination thereof. For example, an extratumoral treatment may comprise applying to the surgical site, and/or an area surrounding a surgical site, a composition/combination of the present disclosure. In this context, haloperoxidase may be administered in solution or in any other dosage form, such as a subcutaneous injection or deposit.

Intratumoral treatment may comprise direct injection into a tumor or a blood vessel supplying a tumor a composition/combination of the present disclosure.

In either non-surgical or surgical anticancer treatment using compositions, combinations or methods of the present disclosure, a patient in need may be treated with a further anticancer therapy, such as an immunotherapy, chemotherapy and/or radiotherapy. The further anticancer therapy may be administered to the patient prior, concurrently or post treatment with the compositions/combinations of the present disclosure.

Further, a person skilled in the art will recognize that administration of haloperoxidase in compositions, combinations or methods of the present disclosure, will provide microbicidal benefits at the site of treatment, in addition to anticancer benefits. The choice of whether to administer, for example, activated haloperoxidase, or a combination of inactive haloperoxidase and peroxide producing oxidase, halide and substrate for the oxidase, will be within the remit of the person skilled in the art and may depend on numerous factors including the type and site of cancer treatment, and/or the degree of control of haloperoxidase activity required.

Further examples of the invention are described below. However, it should be noted that the invention should not be limited to these examples, and that the invention is susceptible to variations, modifications and/or additions other than those specifically described, and it is to be understood that the invention includes all such variations, modifications and/or additions which fall within the scope of the claims.

### EXAMPLES

### Example 1—In Vivo Tumor Reduction Activity of Porcine Eosinophil Peroxidase (pEPO)

An experiment was performed to determine the tumor reduction activity of porcine eosinophil peroxidase (pEPO) in a mouse subcutaneous xenograft.

### Materials

### Tumor Cell Line Description

HT-1080 cells (Cat #CCL-121) purchased from American Type Culture Collection (ATCC) were used for the experiments. The cells were grown in complete media as described below. Cells were seeded in cell culture flasks and incubated at 37° C. in a fully-humidified atmosphere with 5% CO₂. Once the cells reached confluence, they were propagated and/or preserved as described below:

Cell Propagation and Preservation Procedure: performed according to standard methods:
Subculturing Ratio: 1:4 to 1:8
Medium Renewal: 2 to 3 times per week
Propagation Procedure: Removed medium and washed twice with D-PBS (1×). Added 1× trypsin solution and allowed the flask to sit at room temperature (RT) or at 37° C. until the cells detached. Added fresh medium, aspirated and dispense into new flasks. Record the number of passages.
Preservation Procedure: Froze the cells in 95% complete growth medium supplemented by 5% DMSO. The first medium to be used after thawing the cells was Eagle's MEM that was supplemented by adequate concentrations of glucose (4500 mg/l D-glucose).
Passaging Procedure: Removed medium and washed twice with D-PBS (1×) Added 1× trypsin solution and allowed the flask to sit at room temperature (RT) or at 37° C. until the cells detached. Added fresh medium, aspirated and dispense into new flasks. Record the number of passages.
Final Harvest Procedure: 6× T-150 flasks were harvested by removing the medium and washing once with 1×D-PBS. Added 1× trypsin solution and allowed flasks to sit at room temperature (RT) until cells detached. Quenched the trypsin using culture media (with bovine serum) and spun cells at 1000 rpm for 10 min. Washed twice with 1×D-PBS and resuspended the pellet in 1×D-PBS to a concentration of 52×10⁶ cells/ml D-PBS.

HT-1080 cell line propagation, harvest and viability assessment was performed prior to injection into animals revealing the following:
1. Total cell counts: 78×10⁶ cells
2. Cell viability prior to injection: 98%
3. Viable cells/mL: 51×10⁶ viable cells/ml

### Tumors

For the subcutaneous (SC) tumor growing model, a dose of 5.1×10⁶ cells/mouse was injected SC into the right flank of each mouse in a 100 μl volume on study day 1.

Tumor growth was followed twice a week by caliper measurements to determine the three parameters of length, width and height. Tumor volume was calculated according to the formula for an ellipsoid:

4/3π×(La/2)×(Wa/2)×(Ha/2)

La, Wa, and Ha, are the length, width and height of the tumor measured in vivo minus the skin thickness. Bi-fold skin thickness was subtracted from the length and width parameters and single fold skin thickness was subtracted from the height measurement to determine La, Wa and Ha.

### Animal Description

Charles River athymic nude (Nu/Nu) mice (male) were purchased from Charles River Laboratories. Animals were allowed 5 days to acclimate before commencement of the study. Animals were weighed one day prior to injection. Starting body weights were between 20 and 25 grams. Animals were ear punched for identification and housed 5 per cage until randomization by tumor size. Once animals were assigned to groups, they were housed 1 per cage.

### Enzyme Solutions

Compositions of pEPO enzyme solution and activator solution (peroxide) were prepared. The enzyme solution contained a final concentration of pEPO of 2.5 μg/ml, (0.8-0.05 mM each L-alanine, L-proline, glycine, final concentration), ethanolamine (2,4-final concentration), sodium bromide (2 mM) and Tween-80 (0.1%, v/v). The activator solution comprised hydrogen peroxide at 0.003%, v/v, 890 μM in phosphate-buffered saline (PBS) pH 7.4. The vehicle was PBS.

Fifteen microliters of activator solution was added to vehicle or enzyme solution and allowed to incubate for ˜3-5 minutes prior to dosing. Dosing solutions were activated for each individual animal. Following activation, ˜1 ml of dosing solution was injected into the surgical cavity. The cavity was filled to completion until some leaking of fluid from the surgical site was observed.

### Experimental Design

HT-1080 cells fibrosarcoma cells were cultured and expanded under routine conditions noted above. On the day of injection into mice, cells were harvested, washed with phosphate buffered saline, and resuspended at a concentration 5×10⁷ cells/ml, Thirteen athymic nude mice (13 males) were injected SC in the right flank with HT1080 cells (concentration 5.1×10⁶ cells/animal, volume 100 μl/animal). Following injection, animals were weighed weekly and monitored for tumor formation. Tumors were measured twice a week using external calipers once tumors were visible and had reached a measurable size.

When tumors reached 0.5-1 cm³, animals were randomized into two groups of mice. The tumors were surgically removed from both groups. Surgical wounds were sealed after excision of the tumor with surgical glue and then the cavity was filled with dosing solution (˜1 ml/animal). Group 1 received vehicle+activator while Group 2 received of pEPO+activator. Animals were individually housed following surgery. One animal from Group 1 (vehicle+activator) was found dead on the day after surgery. This animal was replaced with an extra tumor bearing animal. The tumor was removed from the replacement animal, the surgical wound sealed and the cavity was treated with 1 ml of vehicle+activator.

After treatment, mortality checks and clinical observation were performed daily. Animals were weighed weekly and on the day of termination. Tumor formation was monitored in animals and when applicable, tumors were measured twice a week using external calipers after tumors had reached a measurable size.

At the end of 4 weeks, animals were euthanized by carbon dioxide followed by cervical dislocation. Tumors were measured with calipers prior to termination. Following termination, tumors were excised, weighed and fixed in 10% neutral buffered formalin,

### Results and Conclusion

Tumor growth and clinical observations are noted in Table 1.

TABLE 1
In vivo Tumor Reduction Activity of pEPO

| | | | |
|---|---|---|---|
| | | | Study Day |
| Animal ID (tag) | Sex | Finding | of Finding |

Group 1: Vehicle plus Activator

| | | | |
|---|---|---|---|
| G1M1 | M | No abnormal findings noted | 1-53 |
| G1M2 | M | No abnormal findings noted | 1-28 |
| | | Tumor regrowth confirmed | 30-39 |
| | | Euthanized due to excess tumor size, tumor collected | 39 |
| G1M3 | M | No abnormal findings noted | 1-15, 22-34, |
| | | Redness on top of tumor | 16-21 |
| | | Slight necrosis on tumor. Trimmed at tumor removal. | 21 |
| | | Tumor regrowth confirmed | 35-43 |
| | | Euthanized due to excess tumor size, tumor collected | 43 |
| G1M4 | M | No abnormal findings noted | 1-21 |
| | | Found dead (replaced with animal G1M4A | 22 |
| G1M4A | M | No abnormal findings noted | 1-53 |
| G1M5 | M | No abnormal findings noted | 1-29 |
| | | Tomor regrowth confirmed | 30-63 |

Group 2: Enzyme plus Activator

| | | | |
|---|---|---|---|
| G2M1 | M | No abnormal findings noted | 1-27, 29-53 |
| | | Open wound (closed with wound clip) | 28 |
| G2M2 | M | No abnormal findings noted | 1-29 |
| | | Tumor regrowth | 30-53 |
| G2M3 | M | No abnormal findings noted | 1-53 |
| G2M4 | M | No abnormal findings noted | 1-53 |
| G2M5 | M | No abnormal findings noted | 1-53 |

Findings exclude the observations of tumor formation prior to surgical removal and treatment

Animals injected with HT-1080 cells developed visible tumors by day 11. Tumor masses increased over time and tumors were surgically removed on study day 21 once tumors had reached an average volume of ˜0.3 cm³ with individual tumors reaching >0.5 cm³ in some animals.

Following tumor removal and treatment, 3 of 5 animals in group 1 (vehicle+activator) had tumor re-growth, although the tumor failed to develop extensively in animal G1M5. Only 1 of 5 animals in group 2 (enzyme+activator) had confirmed tumor re-growth. Animals G1M2 and G1M3 were terminated on study 39 and 43 respectively due to complications arising from the presence of the tumor (difficulty moving, lack of visible eating, lack of feces, overall lethargy). Remaining animals were euthanized on study day 53,

One animal died during the course of this study. Animal G1M4 was found dead on the day after tumor removal surgery and treatment with vehicle+activator. This animal was replaced with one of the remaining tumor bearing animals and treated with vehicle+activator (G1M4a).

Aside from the presence of the tumor, other clinical observations were minor in nature for study animals. IN addition, no clear differences in body weight were noted on the day of study termination between the two groups.

Accordingly, this experiment showed that treatment of tumor cavities with activated pEPO resulted in a decreased number of animals showing tumor regrowth (1 of 5) versus 3 of 5 animals receiving vehicle+activator.

### Example 2—Refined Assessment of the In Vivo Tumor Reduction Activity of pEPO

A similar protocol as described in Example 1 was followed, but with slight variations. Specifically, a larger cohort of thirty-eight athymic nude mice (38 males) were injected SC in the right flank with HT-1080 cells (same concentration as used in Example 1 of 5.0×10⁶ cells/animal, volume 100 μl/animal). Following injection, animals were weighed weekly and monitored for tumor formation, Tumors were measured twice a week using external calipers once tumors were visible and had reached a measurable size.

Whereas in Example 1 tumors were allowed to reach 0.5-1 cm³, in this further example, tumors were allowed to reach 0.1 to 0.3 cm³. Animals were then randomized into two groups of 15 mice. The tumors were surgically removed from both groups. Surgical wounds were sealed after excision of the tumor with surgical glue and then the cavity was filled with dosing solution (H ml/animal). Group 1 received phosphate buffered saline while Group 2 received of pEPO activator. Animals were individually housed following surgery. Procedure according to Example 1 was otherwise followed.

### Results and Conclusion

Tumor growth and clinical observations from this further example are noted in Table 2.

TABLE 2
In vivo Tumor Reduction Activity of pEPO

| | | |
|---|---|---|
| | | Study Day |
| Animal ID | Finding | of Finding |

Group 1: Phosphate Buffered Saline

| | | |
|---|---|---|
| G1M1 | No abnormal finding | 1-99 |
| G1M2 | No abnormal finding | 1-42 |
| | Lesion on skin over tumor. Treated with topical antibiotic | 43-56 |
| | Euthanized due to tumor size | 56 |
| G1M3 | No abnormal finding | 1-99 |
| G1M4 | Lesion on skin over tumor. Treated with topical antibiotic | 21-23 |
| G1M5 | No abnormal finding | 1-99 |
| G1M6 | No abnormal finding | 1-49 |
| | Lesion on skin over tumor. Treated with topical antibiotic | 50-88 |
| | Euthanized due to tumor size | 88 |
| G1M7 | No abnormal finding | 1-27 |
| | Lesion on skin over tumor. Treated with topical antibiotic | 28-31 |
| G1M8 | No abnormal finding | 1-49 |
| | Lesion on skin over tumor. Treated with topical antibiotic | 50-62 |
| | Euthanized due to tumor size | 63 |
| G1M9 | No abnormal finding | 1-65 |
| | Lesion on skin over tumor. Treated with topical antibiotic | 66-92 |
| | Hunched position. Appears dehydrated and losing weight | 91-92 |
| | Euthanized due to moribund status | 92 |
| G1M10 | No abnormal finding | 1-99 |
| G1M11 | No abnormal finding | 1-99 |
| G1M12 | No abnormal finding | 1-49 |
| | Lesion on skin over tumor. Treated with topical antibiotic | 50-66 |
| | Euthanized due to tumor size | 66 |
| G1M13 | No abnormal finding | 1-99 |
| G1M14 | No abnormal finding | 1-99 |
| G1M15 | No abnormal finding | 1-62 |
| | Euthanized due to tumor size | 63 |

Group 2: Enzyme Plus Activator

| | | |
|---|---|---|
| G2M1 | No abnormal finding | 1-49 |
| | Lesion on skin over tumor. Treated with topical antibiotic | 50-90 |
| | Animal appears dehydrated and weak | 91-92 |
| | Euthanized due to tumor size | 92 |
| G2M2 | No abnormal finding | 1-20 |
| | Lesion on skin over tumor. Treated with topical antibiotic | 21-23 |
| G2M3 | No abnormal finding | 1-20, 24-88 |
| | Lesion on skin over tumor. Treated with topical antibiotic | 21-23 |
| | Euthanized due to tumor size | 88 |
| G2M4 | No abnormal finding | 1-93 |
| | Axillary lymph node swollen | 94-99 |
| G2M5 | No abnormal finding | 1-20 |
| | Lesion on skin over tumor. Treated with topical antibiotic | 21-23 |
| G2M6 | No abnormal finding | 1-99 |
| G2M7 | No abnormal finding | 1-42 |
| | Lesion on skin over tumor. Treated with topical antibiotic | 43-98 |
| | Hunched position. Appears dehydrated and losing weight | 98 |
| | Euthanized due to tumor size/moribund status | 98 |
| G2M8 | No abnormal finding | 1-99 |
| G2M9 | No abnormal finding | 1-27, 32-99 |
| | Lesion on skin over tumor. Treated with topical antibiotic | 28-31 |
| G2M10 | No abnormal finding | 1-7, 14-20, 24-99 |
| | Bite marks on animal. Aggressive cage mate removed | 8-13 |
| | Lesion on skin over tumor. Treated with topical antibiotic | 21-23 |
| G2M11 | No abnormal finding | 1-97 |
| | Axillary lymph node swollen | 98-99 |
| G2M12 | No abnormal finding | 1-19, 36-41 |
| | Lesion on skin over tumor. Treated with topical antibiotic | 20-35, 42-85 |
| | Euthanized due to tumor size | 85 |
| G2M13 | No abnormal finding | 1-99 |
| G2M14 | No abnormal finding | 1-30, 32-70 |
| | Opened wound. Closed with clip | 31 |
| | Euthanized due to tumor size | 70 |
| G2M15 | No abnormal finding | 1-99 |

Animals injected with HT-1080 cells developed visible tumors by day 10. Tumor masses increased over time, and tumors were surgically removed on study day 24 once tumors had reached an average volume of ˜0.15 cm³.

Following tumor removal and treatment of the tumor cavity, tumors redeveloped in 6 of 15 animals from Group 1 (phosphate buffered saline) and 5 of 15 animals from group 2 (enzyme+activator). While the number of animals developing tumors and the time to tumor emergence was similar in both groups, the rate of growth of the tumors in the two groups appeared to be different. Tumors in the Group 1 control animals increased in volume more rapidly than those in the Group 2 enzyme+activator treated animals. On average, tumors in group 2 animals required an additional 15 days to reach a similar size compared to group 1 control tumors.

A similar result was observed when monitoring survival of animals. Non-survival was defined as the study day for which the tumor reached maximal tumor volume and the animal was euthanized. As with the tumor volumes, the Enzyme/activator treated animals survived longer than the control treated animals.

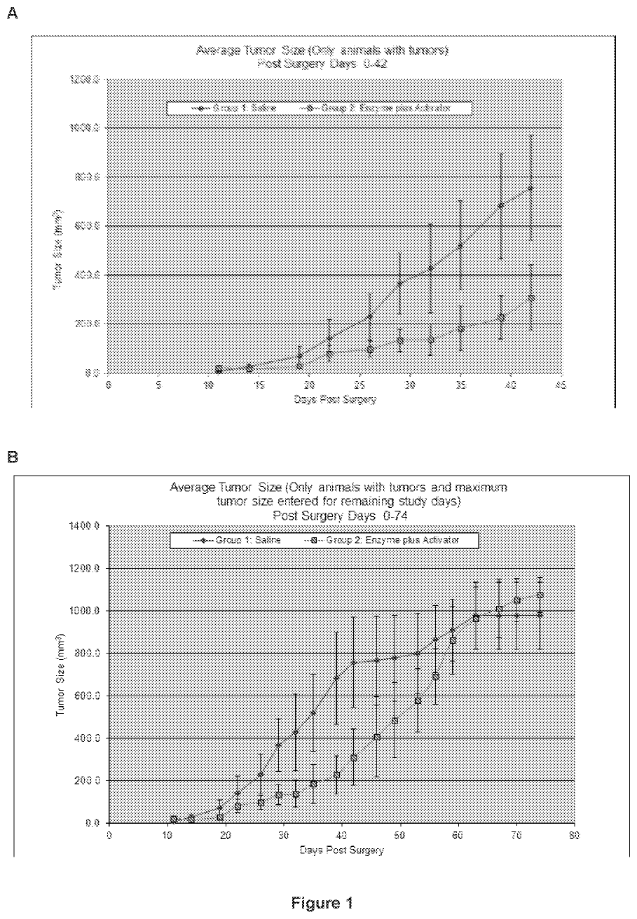

Following surgical removal of HT-1080 tumors and treatment of tumor cavities with activated Enzyme solution (porcine eosinophil peroxidase, pEPO) or phosphate buffered saline as a control, 6 of 15 animals from the phosphate buffered saline group and 5 of 15 animals from Enzyme plus activator group developed tumors. Animals in which tumor cavities were treated with activated enzyme solution resulted in slower tumor growth and extended survival relative to the phosphate buffered saline treatment. Results are shown graphically in FIG. 1, In FIG. 1A, tumor volumes are plotted as a function of post-surgery study day out to study day 42 (the first day any tumor reached the maximum size of 1 cm³). The same data is presented in FIG. 1B except that tumor volumes are plotted out to the terminal study day. When calculating average tumor volumes for later study days, the tumor volume obtained at the time of euthanasia for each animal was used for all remaining study days.

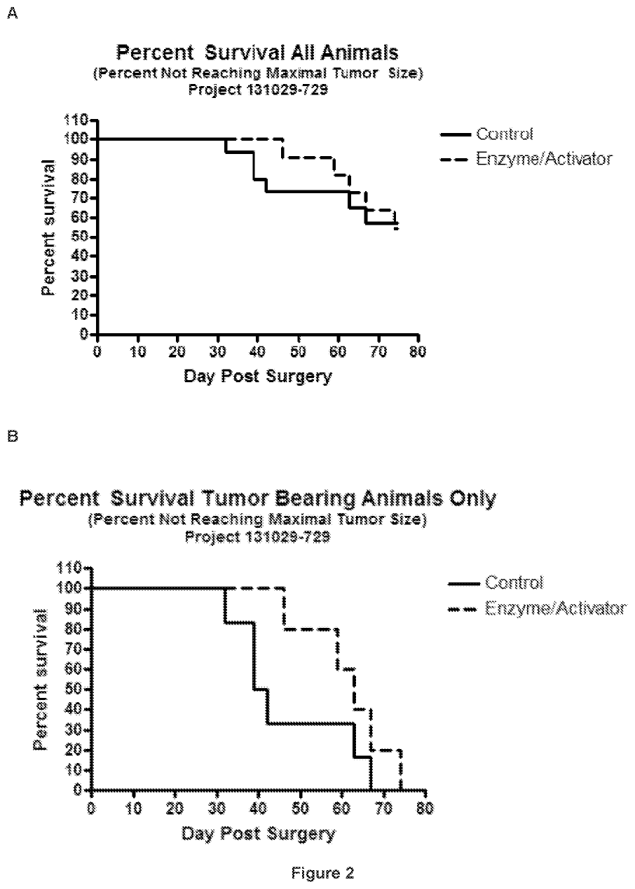

A similar result was observed when monitoring survival of animals. Non-survival was defined as the study day for which the tumor reached maximal tumor volume and the animal was euthanized. Results of the survival study curves are presented in FIG. 2, In FIG. 2A, a survival curve is shown where percent survival is calculated using all study animals. In FIG. 2B, the survival curve is only for the animals that redeveloped tumors. As with the tumor volumes, the enzyme+activator treated animals survived longer than the control treated animals.

### Example 3—Treatment of Bladder Cancer

Treatment of a patient suffering from bladder cancer is envisaged and may encompass one or more of the following:
Maintenance of optimal pH of about 6.0 in the bladder environment by lavage.
Removal of mucosa lining the bladder with dimethyl sulfoxide (DMSO).
Direct instillation/administration of compositions comprising a haloperoxidase, a peroxide or peroxide producing oxidase, and activating compounds, via urinary catheter.
Treatment with a bolus peroxide may comprise 0.3 to 0.03% peroxide with 1 to 10 mEq/L bromide by lavage.

While illustrative embodiments have been illustrated and described, including the best mode known to the inventors for carrying out the invention, those skilled in the art will recognize that the disclosure may be practiced with variations on the disclosed structures, materials, compositions and methods, and such variations are regarded as within the ambit of the disclosure.

Discussion or mention of any piece of prior art in this specification is not to be taken as an admission that the prior art is part of the common general knowledge of the skilled addressee of the specification.

The contents of all references, and published patents and patent applications cited throughout the application are hereby incorporated by reference.

1. A method of treating a solid cancer comprising administering an effective amount of a pharmaceutical composition comprising a haloperoxidase.

2. The method of claim 1, wherein the haloperoxidase is selected from a group consisting of: myeloperoxidase (MPO), eosinophil peroxidase (EPO), lactoperoxidase (LPO), chloroperoxidase (CPO), functional derivatives thereof, and combinations thereof.

3. The method of claim 2, wherein the haloperoxidase is EPO.

4. The method of claim 1, wherein the haloperoxidase catalyzes halide oxidation and disproportionation of peroxide yielding singlet molecular oxygen resulting in one or more of:
inhibition of cancer cell growth,
inhibition of cancel cell metastases, and/or
cancer cell death.

5. The method of claim 4, further comprising administering an effective amount of peroxide or a peroxide-producing oxidase.

6. The method of claim 5, further comprising administering a substrate for the oxidase.

7. The method of claim 6, wherein the peroxide-producing oxidase is glucose oxidase and the substrate is glucose.

8. The method of claim 1, wherein the haloperoxidase is administered with a halide.

9. The method of claim 8, wherein the halide is chloride or bromide.

10. The method of claim 1, wherein the haloperoxidase is administered in a first composition together with at least one further composition comprising one or more of:
a halide,
peroxide or a peroxide-producing oxidase, and
a substrate for the peroxide-producing oxidase.

11. The method of claim 10, wherein said compositions are:
premixed before administration, or
administered concurrently or sequentially.

12. The method of claim 1, wherein the solid cancer is a tumor, preferably a primary or metastatic tumor.

13. The method of claim 12, wherein the haloperoxidase is administered extratumorally or intratumorally.

14. The method of claim 1, wherein the solid cancer is selected from the group consisting of: breast cancer, lung and bronchus cancer, prostate cancer, colon and rectum cancer, melanoma of the skin, bladder cancer, kidney and renal pelvis cancer, endometrial cancer, pancreatic cancer, thyroid cancer, liver cancer, brain cancer and spinal cord cancer.

15. A method of treating a solid cancer in a patient, said method consisting of administering to said patient an effective amount of:
a haloperoxidase, and optionally one or more of:
a halide,
a peroxide or peroxide producing oxidase,
a substrate for said oxidase, and a
a pharmaceutically acceptable carrier.

16. A combination for treating a solid cancer in a patient, said combination comprising:
a haloperoxidase, and at least one of
a halide, and
peroxide or a peroxide producing oxidase.

17. The combination of claim 16, wherein the haloperoxidase catalyzes halide oxidation and disproportionation of peroxide yielding singlet molecular oxygen resulting in one or more of:
inhibition of cancer cell growth,
inhibition of cancel cell metastases, and/or
cancer cell death.

18. The combination of claim 16, which is formulated in a composition that is for administration to said patient.

19. The combination of claim 16, which is formulated in at least two compositions, and wherein said compositions are:
premixed for administration to said human or animal subject, or
for administration concurrently or sequentially to said human or animal subject.

20. The combination of claim 16, wherein the haloperoxidase is selected from a group consisting of: myeloperoxidase (MPO), eosinophil peroxidase (EPO), lactoperoxidase (LPO), chloroperoxidase (CPO), functional derivatives thereof, and combinations thereof.

21. The combination of claim 16, wherein the peroxide-producing oxidase is glucose oxidase.

22. The combination of claim 16, wherein the halide is chloride or bromide.

23. The combination of claim 16, comprising from about 1 μg/ml to about 50,000 μg/ml of haloperoxidase.

24. The combination of claim 16, wherein the peroxide-producing oxidase generates from 100 pmol to 50 μmol peroxide per ml per minute when in the presence of a substrate for the oxidase.

25. The combination of claim 16, wherein the solid cancer is a tumor, and wherein the combination is formulated for administration extratumorally or intratumorally.

26. The combination of claim 16, wherein the solid cancer is selected from the group consisting of: breast cancer, lung and bronchus cancer, prostate cancer, colon and rectum cancer, melanoma of the skin, bladder cancer, kidney and renal pelvis cancer, endometrial cancer, pancreatic cancer, thyroid cancer, liver cancer, brain cancer and spinal cord cancer.

27. A combination for treating a solid tumor in a patient, said combination consisting of:
a haloperoxidase,
a halide, and
peroxide or a peroxide producing oxidase, and optionally
a substrate for said oxidase, and
a pharmaceutically acceptable carrier.

28. A composition for treating a solid cancer in a patient, said composition comprising:
a haloperoxidase, and optionally one or more of:
a halide,
peroxide or a peroxide producing oxidase,
a substrate for said oxidase, and
a pharmaceutically acceptable carrier.

29. The composition of claim 28, wherein the haloperoxidase is selected from a group consisting of: myeloperoxidase (MPO), eosinophil peroxidase (EPO), lactoperoxidase (LPO), chloroperoxidase (CPO), functional derivatives thereof, and combinations thereof.

30. The composition of claim 28, wherein the haloperoxidase catalyzes halide oxidation and disproportionation of peroxide yielding singlet molecular oxygen resulting in one or more of:
inhibition of cancer cell growth,
inhibition of cancel cell metastases, and/or
cancer cell death.

31. The composition of claim 28, in which is the haloperoxidase is formulated together with a peroxide producing oxidase.

32. The composition of claim 28, wherein the peroxide-producing oxidase is glucose oxidase.

33. The composition of claim 28, comprising from about 1 to about 50,000 μg/ml of haloperoxidase.

34. The composition of claim 28, wherein the peroxide-producing oxidase generates from 100 μmol to 50 μmol peroxide per ml per minute when in the presence of a substrate for the oxidase.

35. The composition of claim 28, wherein said composition comprises about 10 to about 5,000 μg/ml of haloperoxidase, and from about 1 to about 500 U/ml of glucose oxidase.

36. The composition of claim 26, wherein the solid cancer is a tumor, and wherein the composition is formulated for administration extratumorally or intratumorally.

37. The composition of claim 26, wherein the solid cancer is selected from the group consisting of: breast cancer, lung and bronchus cancer, prostate cancer, colon and rectum cancer, melanoma of the skin, bladder cancer, kidney and renal pelvis cancer, endometrial cancer, pancreatic cancer, thyroid cancer, liver cancer, brain cancer and spinal cord cancer.

38. A composition for treating a solid cancer in a patient, said composition consisting of:
a haloperoxidase, and optionally one or more of
a halide,
peroxide or a peroxide producing oxidase,
a substrate for said oxidase, and
a pharmaceutically acceptable carrier.
